(19) United States
(12) Patent Application Publication
(10) Pub. No.: US 2026/0102337 A1
(43) Pub. Date: Apr. 16, 2026

(54) Compositions Of Glycoside and Edible Acid For Enhanced Absorption of Glycoside When Incorporated Into A Consumable Product

(71) Applicant: NutriScience Innovations LLC, Milford, CT (US)

(72) Inventors: Michael Lelah, Arlington Heights, IL (US); Edward B. Dosz, Winfield, IL (US)

(21) Appl. No.: 19/276,660

(22) Filed: Jul. 22, 2025

Related U.S. Application Data
(60) Provisional application No. 63/705,535, filed on Oct. 10, 2024.

(51) Int. Cl. A61K 9/00; A61K 31/7032; A61K 47/12

(52) U.S. Cl. CPC A61K 9/0056; A61K 31/7032; A61K 47/12

(57) ABSTRACT

The present disclosure relates generally to a composition including a glycoside combined with an edible acid to synergistically enhance the rate or speed of dissolution of the glycoside resulting in improved absorption of the glycoside when the composition is incorporated into various consumable products. The present disclosure relates to improved consumable products including, but not limited to, food/beverage products, pet food and nutritional supplements, beverages, pharmaceutical and nutraceutical formulations, whereby, a combination of a glycoside with an edible acid has been added to the consumable product to enhance the rate or speed of dissolution of the glycoside resulting in improved absorption of a glycoside when the consumable product is ingested.

### TECHNICAL FIELD

The present disclosure relates generally to a composition of a glycoside with an edible acid, wherein the composition is incorporated into various consumable products, including food, beverages, pharmaceuticals and supplements. The present disclosure further relates to compositions and methods utilizing various concentrations of edible acids to synergistically shorten the dissolution time of the glycoside resulting in improved absorption of the glycoside when ingested. Specifically, the present disclosure relates to a composition of a glycoside, such as salidroside, combined with an edible acid, and incorporating that composition into a consumable product including, but not limited to, human and pet food, human and pet nutrition supplementation, beverages, pharmaceutical and nutraceutical formulations, whereby, the composition provides a shortened dissolution time of the glycoside resulting improved absorption of the glycoside upon consumption of the consumable product.

### BACKGROUND

Glycosides are bioactive compounds found in many plant and animal species. Glycosides consist of a glycone part (which can be a sugar moiety) and an aglycone part (typically the bioactive portion) linked together through a glycosidic bond. Glycosides have many bioactive properties and are used in orally consumed dietary or food supplements, pharmaceuticals and beverages to impart health or medical benefit. The problem with incorporating glycosides in an orally consumed product is that only a small amount of these compounds may quickly dissolve in the gastrointestinal fluid when digested, thus making for either the need for a large dose, which may be uneconomical, or a result in a reduced benefit at a commercial dose. Thus, there is a need to enhance the adsorption of a glycoside in a consumable product to achieve the health and/or nutritional benefits of the glycoside.

A number of methods for enhancing drug and supplement absorption by increasing solubility and by other means have been proposed and are known. Such methods include the use of surfactants, for example, non-ionic surfactant polyethylene glycol combinations with various solvents including alcohols, vitamins, such as vitamin E succinate, and edible acids such as malic acid, in combination with the supplement or drug. Other methods for enhancing absorption by physical means include freeze drying and spray drying. Another approach utilizes the solubility of a weakly basic bioactive which increases with a decrease in the pH below its basic pKa and that of a weakly acidic bioactive which increases with an increase in pH beyond its acidic pKa.

However, some of these methods are not suitable for use in foods, including surfactants, solvents or polyethylene glycol, and some methods are not very effective in optimally improving solubility. Therefore, there is a need for additional improved methods for improving the solubility of a drug or supplement.

Salidroside is a bioactive glycoside found primarily in the plant Rhodiola, also known as golden root or Arctic root. Rhodiola is an adaptogenic herb that has been used in traditional medicine, particularly in Europe and Asia, for its potential health benefits, including enhancing physical and mental performance, reducing fatigue, increasing resilience, increasing low oxygen tolerance, anti-aging, longevity, and combating stress. Rhodiola is also used as a dietary supplement. Salidroside may be extracted from the rhodiola plant or made by fermentation or chemical synthesis.

The potential issue with incorporating salidroside in an orally consumed product is that only a small amount of this compound can be readily dissolved in the gastrointestinal fluid when digested. Salidroside is metabolized and absorbed in the small intestine. This absorption can be impacted by numerous factors, presence of other foods, pH, fat content, intestinal health, drug interactions, chronic diseases, etc. All of these factors can affect gut motility, gastric emptying times, and enzymatic function, which in turn, may negatively impact the absorption of salidroside.

The present disclosure describes the use of an edible acid, such as malic acid in a composition with a glycoside, such as salidroside, to optimize the rate of dissolution of salidroside, thereby increasing its absorption upon entering the small intestine. Additionally, this combination effectively decreases the pH of the small intestine and increases gut permeability, further enhancing the absorption of salidroside. It is known that salidroside is absorbed in the small intestine after being deglycosylated into its aglycone, p-tyrosol, by β-glucosidases. Malic acid also plays a role in the Krebs cycle, which can enhance cellular energy production in gut epithelial cells, supporting positive changes in the gut microbiome. When functioning optimally, the gut microbiome can produce a variety of glycosidases necessary for the hydrolysis and metabolism of salidroside.

Therefore, a need exists for a composition and method to enhance the rapid absorption of a glycoside, such as salidroside, when the glycoside is incorporated into a consumable product, such as a food, beverage, nutraceutical, or pharmaceutical product.

A need further exists for a composition and method to maximize the rate or speed of dissolution to minimize dissolution time and increase absorption of a glycoside, such as salidroside by combining it with malic acid in order to maximize absorption of the glycoside when the composition is added to a consumable product.

### SUMMARY

The present disclosure relates generally to a composition incorporating a glycoside for use in consumable products, including food, beverages and supplements, and methods and compositions utilizing various concentrations of edible acids to synergistically enhance rapid absorption of the glycoside.

The present disclosure further relates to compositions for use in consumables whereby an edible acid, such as malic acid, is combined with a glycoside and the combination added to a consumable product wherein the combination of the glycoside with the malic acids increases the speed or rate of dissolution of the glycoside in the consumable product thereby enhancing absorption of the glycoside when the consumable product is ingested. Examples of consumable products include food products, beverages, pharmaceutical products, dietary ingredients or nutraceutical products for both humans and animals.

In one embodiment, the present disclosure provides a composition for use in a consumable product, the composition comprising a glycoside combined with an edible acid, wherein the combination of the glycoside with the edible acid is configured to provide a synergistic effect to enhance the rate or speed of dissolution of the glycoside when the consumable product is ingested.

In another embodiment, the present disclosure provides a composition for use in a consumable product, the composition comprising a glycoside and an edible acid, wherein the glycoside and edible acid are combined in a ratio of about 1:1.5 to 1:3.0 and, wherein the combination of the glycoside with the edible acid is configured to provide a synergistic effect to maximize the rate or speed of dissolution of the glycoside when the consumable product is ingested resulting in improved adsorption of the glycoside.

In yet another embodiment, a method for enhancing the rate or speed of dissolution of a glycoside in a consumable product is provided. The method comprises the steps of providing a glycoside, combining the glycoside with an edible acid, adding the combination of glycoside and edible acid to a consumable product, wherein the combination of the glycoside with the edible acid is configured to provide a synergistic effect of increasing the absorption of the glycoside when the consumable product is ingested by a mammal.

### DETAILED DESCRIPTION

The present disclosure is based on the multifaceted benefits of edible acids, such as malic acid, in enhancing the absorption of a glycoside via multiple pathways in the small intestines when the glycoside and edible acid are combined into a composition for use in a variety of consumable products, including foods, beverages, nutraceuticals, and pharmaceutical products. In the present disclosure, the preferred glycoside is salidroside, which has multiple potential health benefits, including improving the body's ability to handle stress and reduce fatigue, neuroprotective effects including supporting cognitive function, antioxidant and anti-inflammatory properties, support for cardiovascular health, all of which provide anti-aging benefits and longevity.

The use of an edible acid, such as malic acid to enhance absorption by improving solubility is known. However, such use does not account for or involve combining the acid in an optimized ratio specifically formulated to maximally increase the rate of dissolution of the glycoside for use in a consumable product. This enhancement of a shortened dissolution time can improve absorption when salidroside passes into the small intestines. The inclusion of an edible acid, such as malic acid, supports the activation of glucosidase enzymes in the small intestine, which have an optimum pH between 5.5 and 6.5, ensuring optimal salidroside metabolism to p-tyrosol. Finally, malic acid through its role in the Krebs cycle can enhance cellular energy production in gut epithelial cells, supporting microbial diversity and function, such as enzyme production, which can perpetuate further metabolism and absorption of salidroside.

The present disclosure provides a composition for use in a consumable product including a food, beverage, pharmaceutical/nutraceutical product, wherein the composition incorporates an amount of edible acid, such as malic acid, to synergistically maximize the rate or speed of dissolution of salidroside, which can then lead to enhanced and more rapid absorption of the salidroside when the consumable product is ingested. The advantage and result of the present composition is enhanced absorption through rapid solubility of the salidroside which is necessary since the gut of a mammal is a fast-moving flow-through system. Without being bound by theory, it is believed the enhanced absorption is accomplished via a disruption in the mucus layer that lines the small intestines, where salidroside is absorbed, thus causing an increase in gut permeability. The solubility of a weakly basic bioactive increases with a decrease in the pH below its basic pKa and the solubility of a weakly acidic bioactive increases with an increase in pH beyond its acidic pKa, is known.

The present disclosure provides an unexpected optimum maximum rate or speed of dissolution for salidroside when combined with malic acid, as determined by a minimum dissolution time, and occurring at a pH below the pKa for salidroside (Examples 1 and 2 below). This minimum dissolution time is governed by the ratio of salidroside to malic acid, and deviations from this ratio, either higher or lower, lead to an undesirable increase in dissolution time. The existence of a minimum dissolution time, at a pH less than the pKa of salidroside, is unexpected. This shortened dissolution time would be critical for rapid solubilization in the intestinal fluid and for subsequent absorption into the gut, where the constant motility challenges the uptake of the salidroside. In addition, even though the measured pH (Example 1 below) is less than that of the pKa of salidroside (pKa of salidroside is 9.98+/−0.15 which is a weak base), there is no previously known optimum minimum dissolution time for such a combination.

### EXAMPLES

Example 1: Dissolution time for salidroside powder in the presence of L-malic acid. Solvent used was purified water at 22° C. Results of the dissolved solutions are shown in Table 1 below.

TABLE 1

| | | Salidroside/ | | | |
|---|---|---|---|---|---|
| | L-Malic | L-malic | | | |
| Salidroside | Acid | acid | Water | Dissolution | pH after |
| (mg) | (mg) | ratio | (mL) | (sec) | dissolution |
| 15.0 | 0.0 | ND | 2.0 | 62 | 4.9 |
| 15.0 | 5.0 | 3.0 | 2.0 | 25 | 4.8 |
| 15.0 | 10.0 | 1.5 | 2.0 | 25 | 2.4 |
| 15.0 | 15.0 | 1.0 | 2.0 | 22 | 2.5 |
| 15.0 | 20.0 | 0.75 | 2.0 | 28 | 2.4 |
| 15.0 | 35.0 | 0.43 | 2.0 | 30 | 2.4 |
| 15.0 | 85.0 | 0.18 | 2.0 | 50 | 2.4 |

In this example, Table 1 illustrates there is an optimal shortest dissolution time unexpectedly observed when the salidroside to L-malic acid ratio is about 1:1.0-1:3.0. The measured pH's are unrelated to the known pKa for salidroside of 9.98+/−0.15.

Example 2: Dissolution time for salidroside powder in the presence of L-malic acid. Solution used was gastric fluid USP-2.0 g NaCl, 0.32 g pepsin enzymes (equivalent to 1300 units/mg). pH adjust with HCl to 1.2, in 1.000 ml purified water at 37° C. Results of the dissolved solutions are shown in Table 2 below.

TABLE 2

| | | Salidroside/ | | | |
|---|---|---|---|---|---|
| | L-Malic | L-malic | Gastric | | |
| Salidroside | Acid | acid | Fluid | Dissolution | pH |
| (mg) | (mg) | ratio | (mL) | (sec) | (adjusted) |
| 15.0 | 0.0 | ND | 2.0 | 61 | 1.2 |
| 15.0 | 5.0 | 3.0 | 2.0 | 45 | 1.2 |
| 15.0 | 10.0 | 1.5 | 2.0 | 58 | 1.2 |
| 15.0 | 15.0 | 1.0 | 2.0 | 68 | 1.2 |
| 15.0 | 20.0 | 0.75 | 2.0 | 72 | 1.2 |
| 15.0 | 50.0 | 0.30 | 2.0 | 98 | 1.2 |
| 15.0 | 85.0 | 0.18 | 2.0 | 115 | 1.2 |

In this example, an optimum minimum dissolution time was unexpectedly observed and occurred when the ratio of salidroside to L-malic was about 1:3.0.

The results from these experiments demonstrate an optimum (shortest) dissolution time dependent on the ratio of salidroside to L-malic acid. In contrast dissolution times are longer at both higher and lower ratios of L-malic acid. Given the gastrointestinal tract functions as a flow-through system, residence time in the various compartments, especially the small intestine, can be short, thus shorter dissolution times are an important parameter for improved absorption of potentially beneficial components and their resulting health benefits.

Specifically, a shorter dissolution time means greater opportunity for enhanced absorption by the digestive system to provide the health benefits of the glycoside discussed above. Thus, the advantages of the present disclosure include: (a) the use of an edible acid such as malic acid specifically in conjunction with/combined with a glycoside, such as salidroside to maximize the speed or rate of dissolution or solubilization, leading to enhanced absorption of the glycoside, and (b) an unexpected finding of an optimum, minimum, shortest dissolution time of the combination of an edible acid and glycoside, thereby further enhancing the health benefit of the composition.

As discussed, the compositions of salidroside combined with malic acid have the shortest dissolution time of salidroside when the ratio of salidroside to L-malic acid is about 1:1.0 to 1:3.0. This means for a human daily dose of, for example, 15 mg salidroside, an optimum amount of L-malic acid combined with salidroside for improved solubilization would be about 15 mg to about 45 mg. The daily human dose of salidroside is not limited to 15 mg but can be any dose with a health benefit or an expected health benefit, for example between about 5 mg and about 100 mg of salidroside.

Glycosides useful in the compositions of the present disclosure include a variety of different glycosides, including salidroside (from rhodiola), withanolide glycosides (from ashwagandha), amygdalin and prunasin (from bitter almond), dhurrin (from sorghum) and linamarin and lotaustralin (from barley, flax, white clover, and cassava). Edible acids useful in the compositions of the present disclosure include a variety of suitable edible acids and/or combinations of edible acids, such as acetic acid, DL-malic acid, L-malic acid, citric acid, lactic acid, phosphoric acid, tartaric acid, etc.

Compositions of the glycoside and edible acid of the present disclosure can be combined through various known methods, including a physical combination/blending of powders, co-spray drying or fluidized bed drying with agglomeration to produce a single particle, unified compositions, or coatings to form beadlets, microbeadlets or other forms. The present compositions can include delayed release or extended-release formulations, such as using enteric coatings or delayed release capsules, so that stomach dissolution is bypassed and dissolution of the composition only occurs in the small intestine.

It should be noted that various changes and modifications to the presently preferred embodiments described herein will be apparent to those skilled in the art. Such changes and modifications may be made without departing from the spirit and scope of the present invention and without diminishing its attendant advantages. Further, any references throughout the specification to “the invention” are nonlimiting, and it should be noted that claim limitations presented herein are not meant to describe the invention as a whole. Moreover, the invention illustratively disclosed herein suitably may be practiced in the absence of any element which is not specifically disclosed herein.

1. A composition for use in a consumable product, the composition comprising:
a glycoside combined with an edible acid, wherein the combination of the glycoside with the edible acid is configured to provide a synergistic effect to maximize the rate or speed of dissolution to minimize dissolution time and increase absorption of the glycoside when the composition is incorporated into the consumable product and the consumable product is ingested.

2. The composition of claim 1, wherein the glycoside is salidroside.

3. The composition of claim 2, wherein the edible acid is malic acid.

4. The consumable composition of claim 1, wherein the composition includes a ratio of glycoside to edible acid of about 1:1.5 to 1:3.0.

5. The composition of claim 4, wherein the glycoside and edible acid are combined through one of a physical combination/blending of powders, co-spray drying or fluidized bed drying with agglomeration to produce a single particle, unified compositions, or coatings to form beadlets, microbeadlets or other forms.

6. The composition of claim 1, wherein the consumable products include foods, beverages, supplements, pharmaceuticals and nutraceuticals.

7. The composition of claim 6, wherein the consumable products include products for mammals.

8. The composition of claim 4, wherein the combination of the glycoside with the edible acid is configured to maximize the rate or speed of dissolution of the glycoside when the composition is added to the consumable product and the consumable product is consumed.

9. A composition for use in a consumable product, the composition comprising:
a glycoside;
an edible acid;
wherein the glycoside and edible acid are combined in a ratio of about 1:1.5 to 1:3.0; and,
wherein the combination of the glycoside with the edible acid is configured to provide a synergistic effect to shorten the rate or speed of dissolution of the glycoside when the composition is added to a consumable product and the consumable product is ingested.

10. The composition of claim 9, wherein the combination of the glycoside with the edible acid provides the synergist effect of shortening a time of dissolution of the glycoside when the consumable product is consumed.

11. A method for enhancing the rate or speed of dissolution of a glycoside in a consumable product, the method comprising the steps of:
providing a glycoside;
combining the glycoside with an edible acid,
adding the combination of glycoside and edible acid to a consumable product;
wherein the combination of the glycoside with the edible acid is configured to provide a synergistic effect of maximizing the rate or speed of absorption of the glycoside when the consumable product is ingested by a mammal.

12. The method of claim 11, wherein the glycoside and edible acid are combined at a ratio of 1:1.5 to 1:3.

13. The method of claim 11, wherein the method further includes determining a dissolution time of the glycoside when combined with the edible acid.

14. The method of claim 13, wherein the method further includes determining a minimum dissolution time of the glycoside when combined with the edible acid.

15. The method of claim 14, wherein the minimum dissolution time of the glycoside is configured to enhance the rate or speed of absorption of the glycoside by an intestinal track of the mammal when the consumable product is ingested by the mammal.
